(19) United States
(12) Patent Application Publication
(10) Pub. No.: US 2017/0071332 A1
(43) Pub. Date: Mar. 16, 2017

(54) Height Adjustable Desks with Ultraviolet Light Irradiation of Keyboard Tray

(71) Applicant: Next Technologies, Inc., Georgetown, TX (US)

(72) Inventors: Jason Brian Herring, Georgetown, TX (US); Shane Michael Gilbreath, Georgetown, TX (US)

(73) Assignee: NEXT TECHNOLOGIES, INC., Georgetown, TX (US)

(21) Appl. No.: 14/854,821

(22) Filed: Sep. 15, 2015

(51) Int. Cl. A47B 21/02; A47B 21/03; A61L 2/10; A47B 9/20

(52) U.S. Cl. CPC A47B 21/02; A47B 9/20; A47B 21/0314; A61L 2/10; A47B 2021/0335; A61L 2202/20

(57) ABSTRACT

Height adjustable desks with a keyboard tray system that is irradiated by an ultraviolet light disinfectant unit. The ultraviolet light unit resides parallel to and above the keyboard tray while being rigidly affixed to the underside of the desktop. The ultraviolet light unit also has the ability to irradiate the keyboard tray top when it is extended for typing and/or when it is retracted under the desktop for storage. And the ultraviolet light unit may be manually activated using a power switch located, for example, with the telescopic desk legs user control panel. And/or the ultraviolet light unit may be automatedly controlled, such as by being connected to a motion sensor to detect when a user is within close proximity to the keyboard, and thus turn off the UV light; and/or via a timer mechanism that activates the UV light unit for a regularly scheduled eradiation session.

### FIELD OF THE DISCLOSURE

This invention relates generally to computer furniture and particularly to height adjustable desks. More particularly, this disclosure relates to height adjustable desks with ultraviolet (UV) light irradiation directed onto the keyboard tray to eradicate infections lurking thereon; and (optionally) motion sensors to detect when a user is near the desktop to prevent harmful exposure to the UV rays.

### BACKGROUND OF THE DISCLOSURE

Computer peripherals, such as keyboards, computer mouse(s), and touchpads can be infected with a wide variety of pathogens, depending on their location (e.g. hospital, public café, library, office, home, etc.). Each user may deposit and contract the microbial agents (e.g. bacteria and fungi) that live on top of these computer peripherals. Documented transmission of infections include, for example: methicillin-resistant Staphylococcus aureus, staphylococci and Pseudomonas spp., nosocomial A. baumannii, enterococci and Enterobacter spp.

Ultraviolet light radiation, notably UV-C that is less than or equal to 280 nanometers has been shown to be effective in irradiating a majority of the microbial agents on keyboards. UV-C damages the DNA of the microbial agents so that they are not able to replicate and thus die off. UV-C light emitted at 265 nanometers and at about 185 nanometers have been shown to be the most effective at eradicating microbial agents, but it may also be damaging to human DNA, so care must be taken to not expose the user to the ultraviolet light.

The prior art discloses standard office desks with computer keyboard disinfecting systems comprising ultraviolet light emitting units. For example, CN201668109, published Dec. 15, 2010, and entitled “Keyboard-mouse automatic disinfecting computer desk”, discloses a standard office desk with an ultraviolet light above the keyboard that is on a timer. And, the outer and inner end surfaces of the keyboard and mouse trays are provided with ultraviolet sheltering pieces to protect the user from being exposed to the ultraviolet light rays. These sheltering pieces do not appear to be able to protect the user if they are typing when the ultraviolet light is on, so the user must remember to turn the UV lights off when they are working at the computer.

U.S. Pat. No. 6,278,122, published Aug. 21, 2001 to Gagnon, and entitled “Keyboard and Mouse Sterilizing Device”, discloses an enclosure with a door for the keyboard to slide into. A safety switch prevents the ultraviolet light from turning on until the keyboard is stored within the enclosure and the door is closed. Therefore, the user must remember to store the keyboard in order for it to be disinfected.

Likewise, U.S. Patent Application 20080067417, published Mar. 20, 2008 to Lane et al, and entitled “Self-Sterilizing Input Device” teaches an enclosure for irradiating the keyboard within an enclosure with a motorized mechanism to move the keyboard tray into and out of the enclosure. The user may activate the motor from the computer screen via software.

Additionally, irradiation devices are available for standard office desks, but not for height adjustable desks, which have become increasingly popular because of their ability to relieve the adverse effects of sitting for long periods of time. These adverse effects include, for example: an accumulation of metabolites; accelerated disk degeneration leading to disk herniation; premature mortality; chronic diseases such as cardiovascular disease, diabetes, and cancer; and obesity (Pope et al.; Pronk et al.).

Most height adjustable desks also lack keyboard trays, especially ones that extend out from beneath the desktop and retract in. Instead, height adjustable desks primarily comprise a flat desktop that raises and lowers from a sitting position to a standing position using motorized legs, or via the user manually lifting the desktop, or activating other non-motorized mechanisms. Both the computer and keyboard reside on top of this adjustable desktop. Additionally, irradiation mechanisms on height adjustable desks are not used because of the lack of additional structures attached to the moving desktop (i.e. separate enclosures for storing the keyboard in while it is irradiated and keyboard trays with UV shields).

Therefore, there is a need within the office furniture industry for a height adjustable desk with a keyboard and mouse disinfecting unit that does not require significantly modifying the desktop surface with separate enclosures for irradiation units. In the absence of these enclosures, the user should still be completely protected from exposure to the damaging effects of ultraviolet light. And, the user should have the option of not needing to actively take steps to disinfect the keyboard and mouse unit, but rather there should be a disinfecting system that automatically disinfects the keyboard and mouse unit when the user is not within close vicinity of the height adjustable desk.

### SUMMARY OF THE DISCLOSURE

The various embodiments disclosed herein comprise height adjustable desks with a keyboard tray system that is irradiated by an ultraviolet (UV) light unit to disinfect any object residing on the tray. The tray is movable between an extended position that allows the user to type on the keyboard and a retracted position that stores the tray beneath the desktop. The tray does not need to be enclosed, shielded or otherwise contained while it is being irradiated. Instead, the UV light unit comprises mechanisms to prevent the unit from turning on when the user is in close enough proximity to it to be exposed to the UV rays, such as a user control switch and/or a motion sensor switch.

In one or more embodiments, the UV light unit resides parallel to and above the tray while being affixed to the underside of the desktop, such as along the back side of the desktop. In other words, the UV light unit is mounted on the desktop underside and along the desktop backside, and positioned over the keyboard tray system.

Other locations of the UV light unit are within the scope of this disclosure, such as the UV light unit being affixed to the back of, and on top of, the tray (i.e. next to the user) and pointed towards the front of the desktop. In all embodiments, the UV light would cover the entire surface of the tray, and thus disinfect all objects on the tray, such as a keyboard, mouse, mouse pad, etc.

In one embodiment, the UV light can be activated when the keyboard tray is extended and/or retracted. When the keyboard tray is extended and the UV light unit is on, the user is not standing near the tray—e.g. is standing in front of the desktop, or away from the desktop, and as detected by a motion sensor device.

Therefore, in this embodiment, the UV light unit area of exposure covers the tray top when it is extended and when it is retracted under the desktop because the UV light unit is controlled by a motion sensor that detects when the user is near the desk and automatically deactivates the UV light unit.

In another embodiment, the UV light unit only has the ability to irradiate the tray when it is in a retracted position beneath the desktop. Therefore, the UV light rays only point downward and towards the middle of the desktop, and thus the rays are not pointed to any area around the desktop. This configuration would significantly reduce accidental exposure to the user because their hands would not be on the keyboard when the UV light unit turned on. This embodiment does not require a motion sensor light to detect a user's presence. Instead, the UV light unit is deactivated whenever the keyboard tray is extended.

In another embodiment, the UV light unit is automatedly turned on and off. For example, the UV light unit may be on a timer that routinely irradiates the tray at pre-set time periods (e.g. every morning at 3:00 am), or after pre-set conditions are met (e.g. every time the user retracts the tray). Additionally, the automated UV light unit may have a manual over-ride to permit the user to manually turn on and off the UV light unit and block the automated system from activating.

Additionally, the UV light unit may further comprise safety features to protect the user from exposure to the UV light rays, such as a motion sensor to detect when a user is within close proximity to the tray. If detected, then the light unit will automatically shut off to protect the user from the adverse effects of exposure to UV light rays; and/or the light unit will not turn on. The motion sensor may be permanently on, with a manual override as a backup.

Manual Overrides and Activation Switches: And the UV light unit may be under the operational control of a user activator switch residing, for example, on the desktop or on the UV light unit. For example, a user control panel on the desktop may comprise: a first user activated switch that activates and deactivates the UV light unit (e.g. user disinfects tray when s/he remembers to; or as an override for automated activation); and a second power switch that raises and lowers the desktop by extending and retracting the desk legs. This user control panel may further comprise a third switch to manually activate and deactivate a motion sensor device.

Lastly, the height adjustable desks of the present disclosure comprise a flat desktop supported and permanently affixed to a plurality of height adjustable legs (e.g. two legs, four legs, etc.). The desk legs are manually or automatedly (i.e. powered via motors housed or connected to the desk legs) to extend and retract the legs so as to raise and lower the desktop between a sitting position and a standing position.

The height adjustable desks further comprise a keyboard tray system able to slide a keyboard tray from an extended position for typing, to a retracted position stored beneath the desktop. The keyboard tray system comprises: a horizontally mounted keyboard tray large enough to hold a keyboard, mouse, and mouse tray; a bent keyboard arm support affixed to the underside of the keyboard tray, and front to back slidable by being inserted into a rectangular keyboard guide rail; and the rectangular keyboard guide rail mounted via fixation mechanisms on the centerline of the desktop underside to enable the system to move between a frontward and backward direction relative to the desktop.

The various embodiments disclosed herein further comprise a method of disinfecting a keyboard tray on a height adjustable desk, comprising: a. providing a height adjustable desk comprising: a desktop; a plurality of height adjustable desk legs; a keyboard tray system mounted beneath the desktop and slidable between an extended position from a desktop backside and a retracted position beneath the desktop; and an ultraviolet light unit mounted on an underside of the desktop above the keyboard tray system; b. activating the ultraviolet light unit when the user is not near the keyboard tray system or when the keyboard tray system is retracted beneath the desktop; and, c. deactivating the ultraviolet light unit after a sufficient amount of time effective to disinfect the keyboard tray system, or when a user moves near the keyboard tray system.

In one embodiment, the ultraviolet light unit is controlled by a timer switch.

In another embodiment, the ultraviolet light unit is activated whenever the keyboard tray is moved to the retracted position.

In another embodiment, the height adjustable desk further comprises a motion sensor connected to the ultraviolet light unit, and the motion sensor deactivates the ultraviolet light unit whenever the user is detected near the keyboard tray system.

In another embodiment, the height adjustable desk further comprises a user control panel with two switches comprising: a first user switch to activate the telescopic desk legs extension and retraction; and a second user switch to activate and deactivate the ultraviolet light unit. The desk control panel may further comprise a third user switch to activate and deactivate the motion sensor.

### BRIEF DESCRIPTION OF THE DRAWINGS

The above and other features, aspects, and advantages of the present disclosure will become better understood with regard to the following description, appended claims, and accompanying drawings where in FIGS. 1-18, the view is described from the perspective of the user facing backside of the desk, such as if they were sitting at the desk back side and facing towards the opposing desk front side. Therefore, the right side of the desk is the user's right side, and the left side of the desk is the user's left side.

FIG. 1 is a perspective right view of the back side (user side) of a first embodiment comprising a two legged height adjustable desk with the keyboard tray extended and the UV disinfectant unit pointing down on the tray.

FIG. 2 is a perspective left view of the back underside of the two legged desk with the key board tray extended.

FIG. 3 is a top plan view of the desktop with the keyboard tray extended.

FIG. 4 is a left side view with the keyboard tray extended.

FIG. 5 is another top plan view with the keyboard tray extended and showing a projection line “6-6”.

FIG. 6 is a cross-sectional left side view of the two legged desk along the projection line of FIG. 5.

FIG. 7 is an exploded perspective, left side view illustrating the components of the desktop tray system and the UV disinfectant unit.

FIG. 8 is an exploded view of FIG. 2 illustrating the components of the desktop from a perspective left underside view.

FIG. 9 is a magnified view of the keyboard tray, user power activation switches and the overhead UV disinfectant unit.

FIG. 10 is a perspective right view of the back side (user side) of a second embodiment comprising a four legged height adjustable desk with the keyboard tray extended and the UV disinfectant unit pointing down on the tray.

FIG. 11 is a perspective left view of the back underside of the four legged desk with the key board tray extended.

FIG. 12 is a top plan view of the desktop with the keyboard tray extended.

FIG. 13 is a left side view with the keyboard tray extended.

FIG. 14 is a top plan view of the four legged desk with the keyboard tray extended and showing a projection line “14-14”.

FIG. 15 is a cross-sectional left side view of the four legged desk along the projection line of FIG. 14.

FIG. 16 is an exploded perspective, left side view of FIG. 10 illustrating the components of the desktop tray system and the UV disinfectant unit.

FIG. 17 is an exploded view of FIG. 11 illustrating the components of the desktop from a perspective left underside view.

FIG. 18 is an expanded view of the keyboard tray, user activation switches, and the overhead UV disinfectant unit.

### DETAILED DESCRIPTION OF EXEMPLARY EMBODIMENTS

The following description should not be used to limit the scope of the present disclosure. Other examples, features, aspects, embodiments, and advantages of the disclosure will become apparent to those skilled in the art from the following description, which includes by way of illustration, multiple various example embodiments. As will be realized, the disclosure is capable of other different and obvious aspects, all without departing from the inventive subject matter. Accordingly, the drawings and descriptions should be regarded as illustrative in nature and not restrictive. It should therefore be understood that the inventors contemplate a variety of embodiments that are not explicitly disclosed herein.

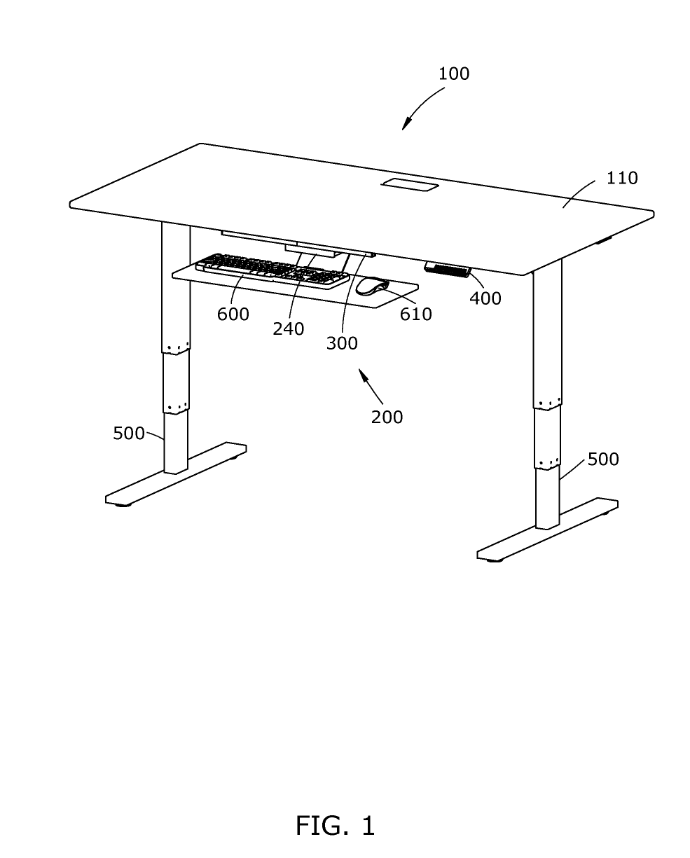

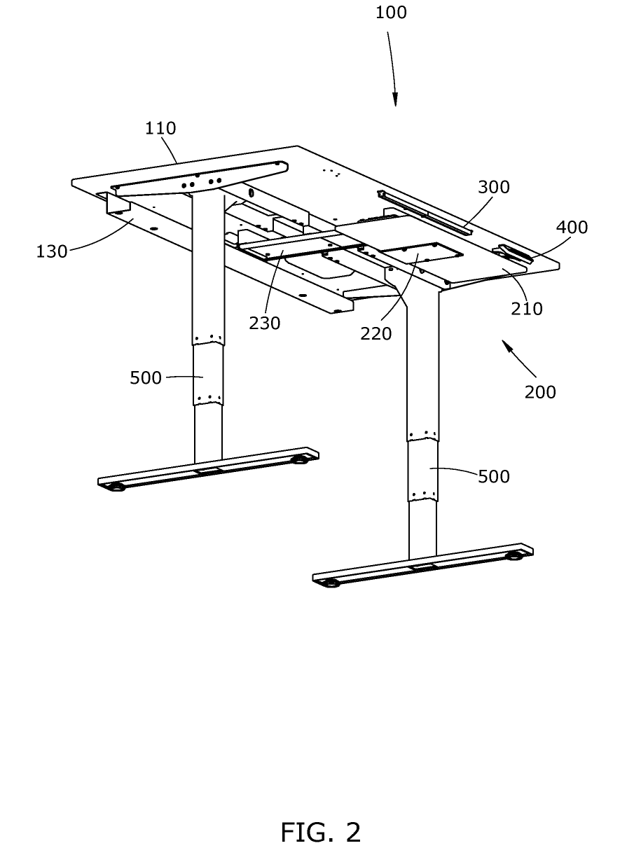

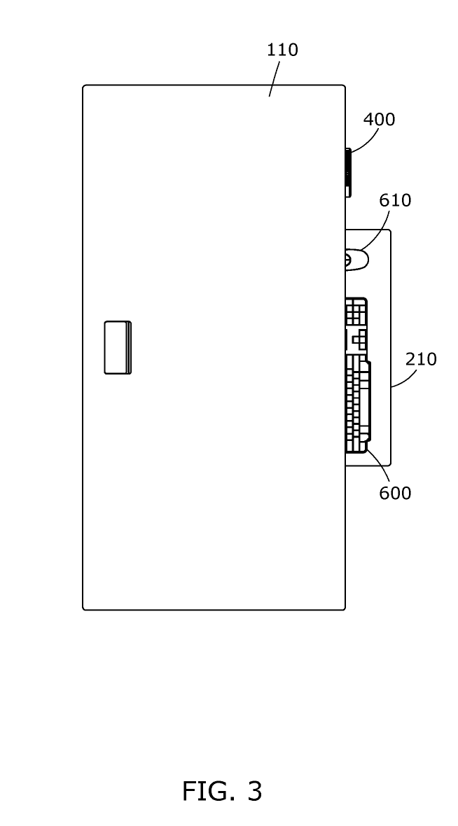

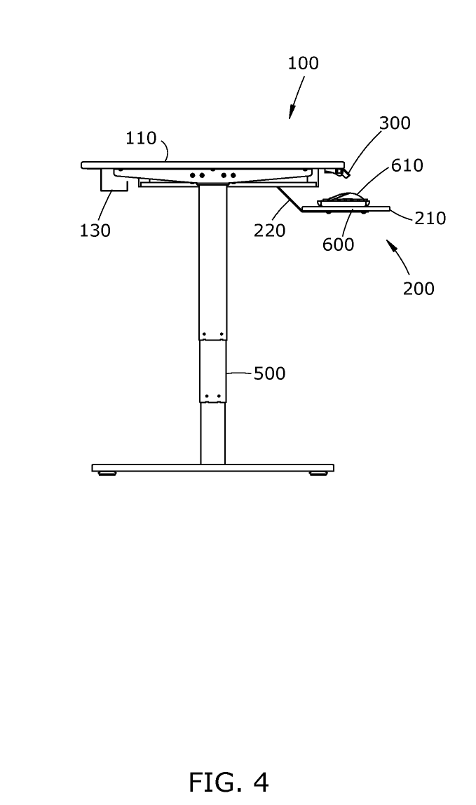

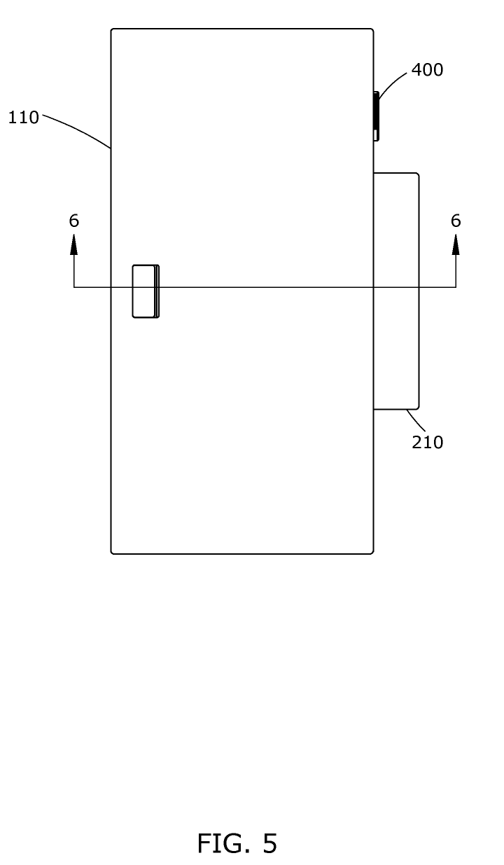

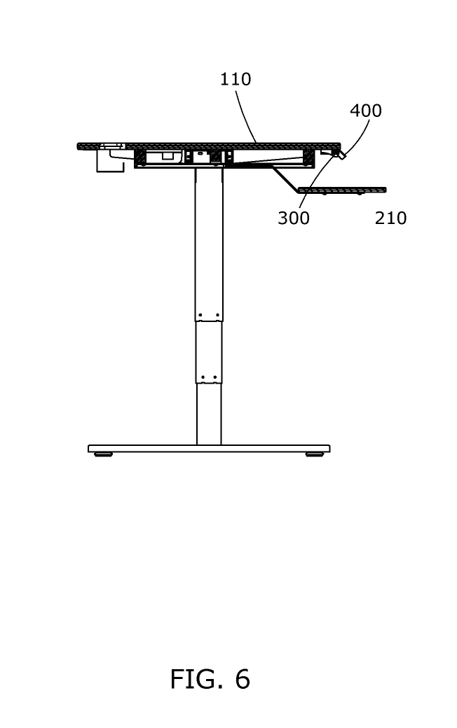

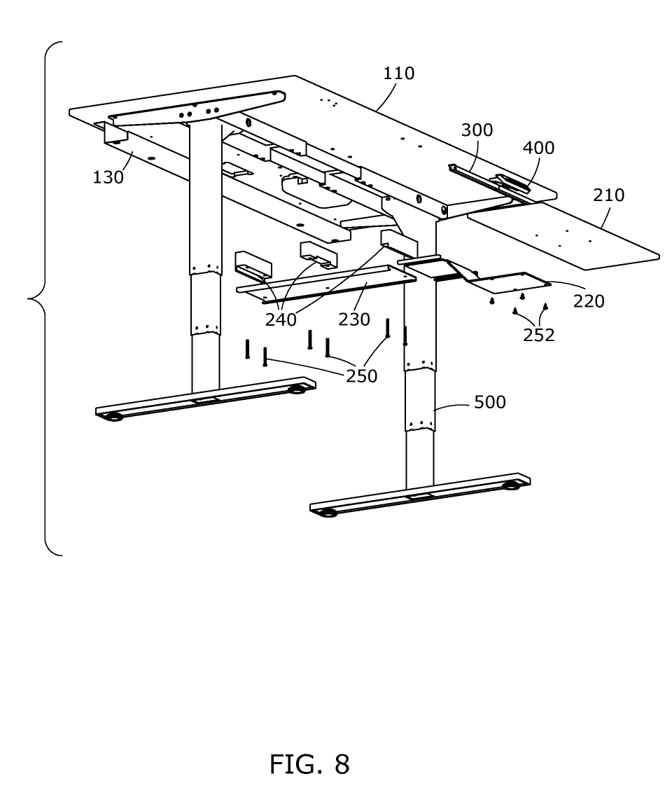

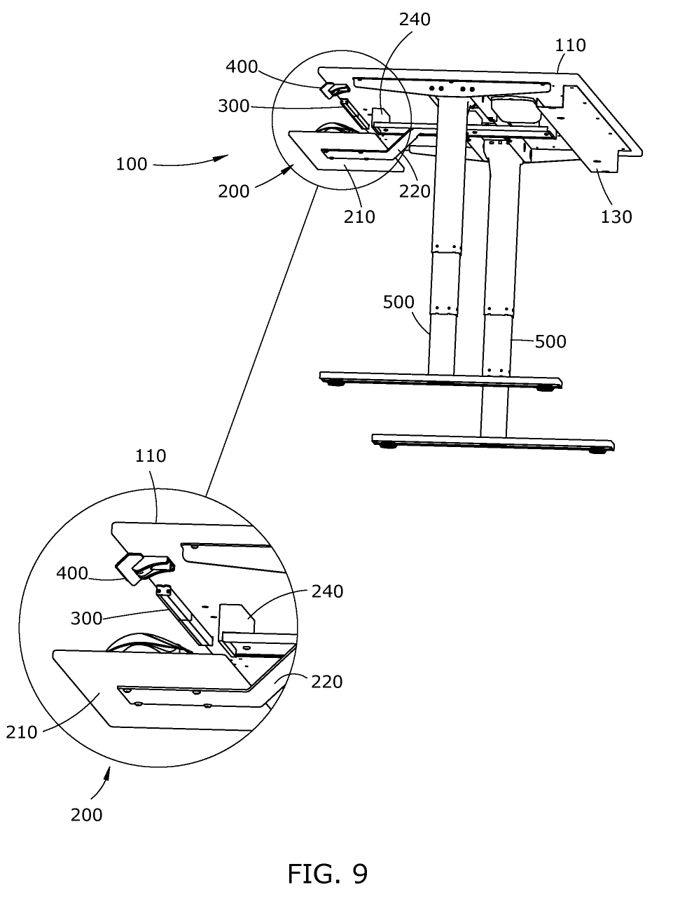

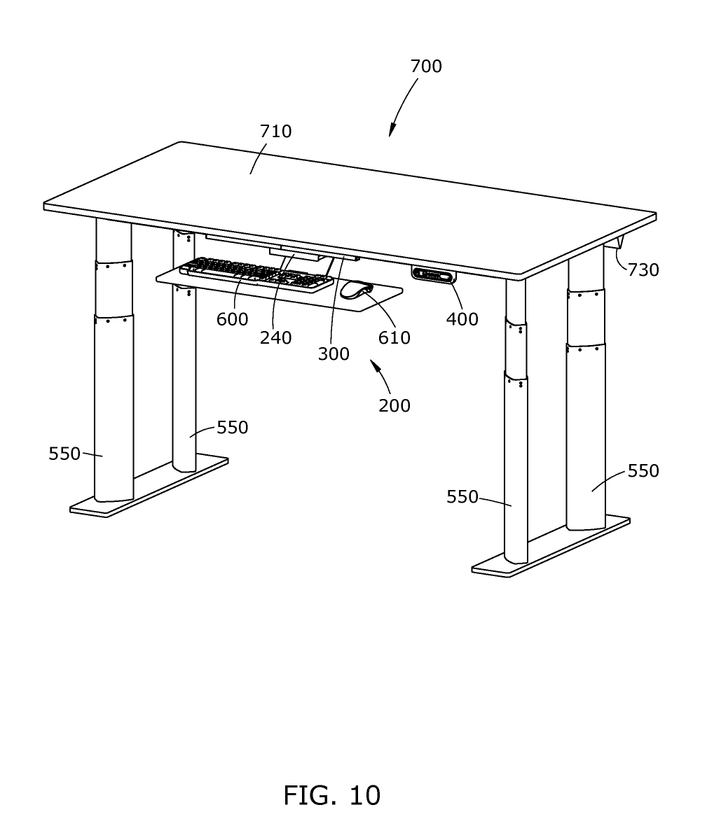

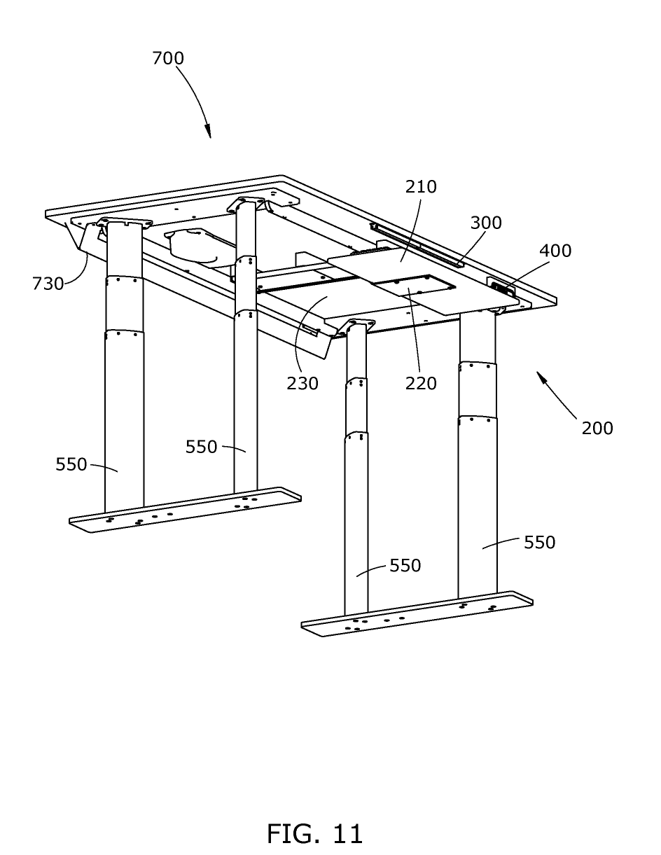

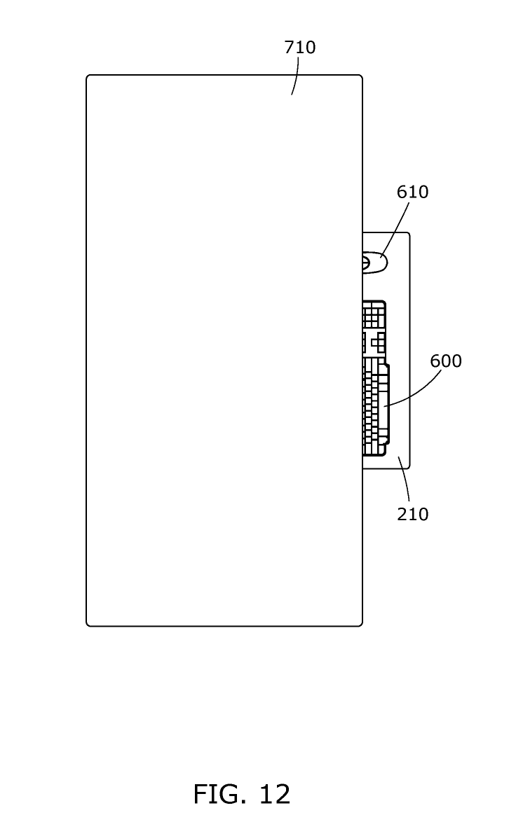

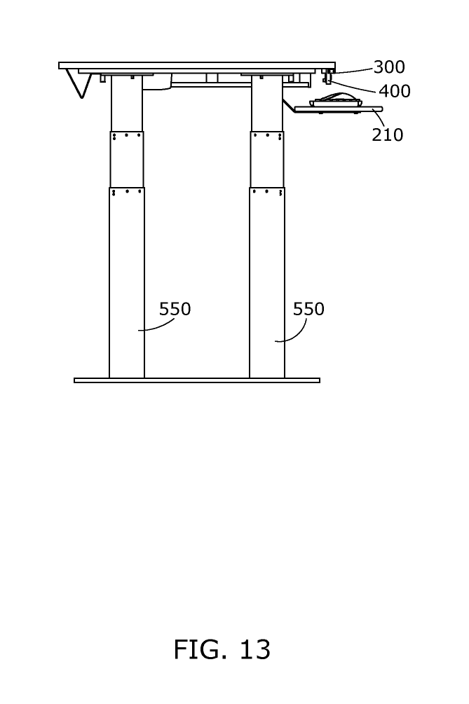

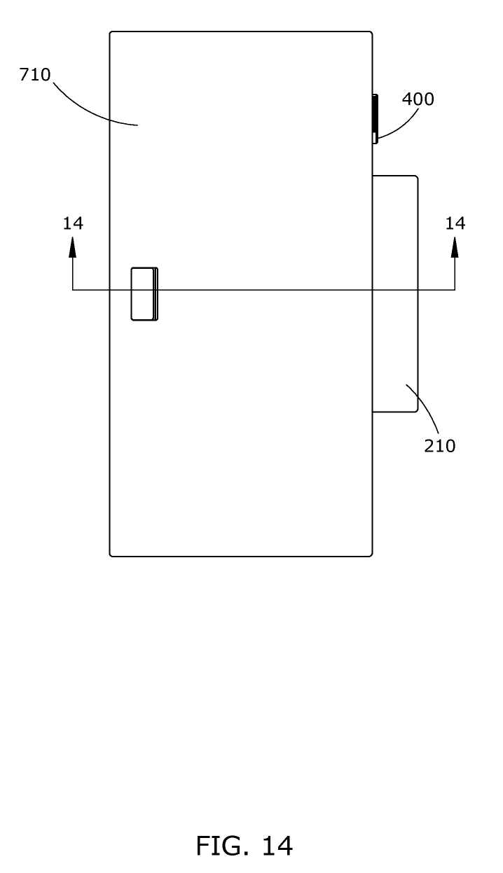

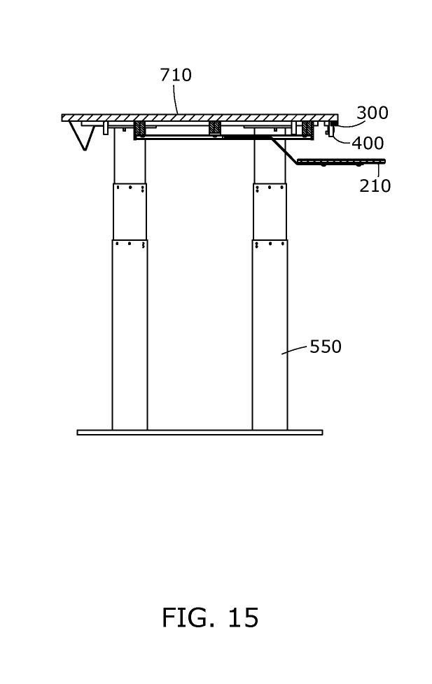

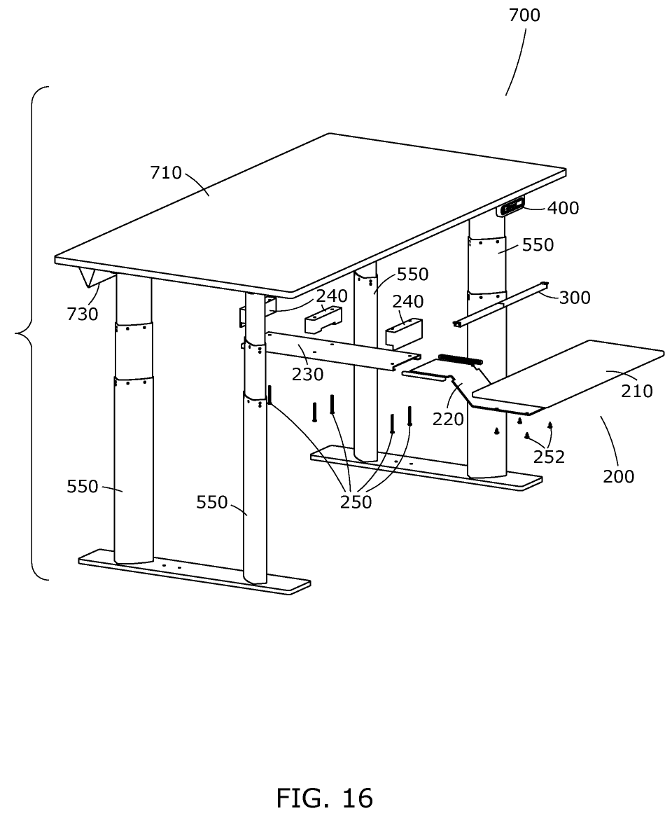

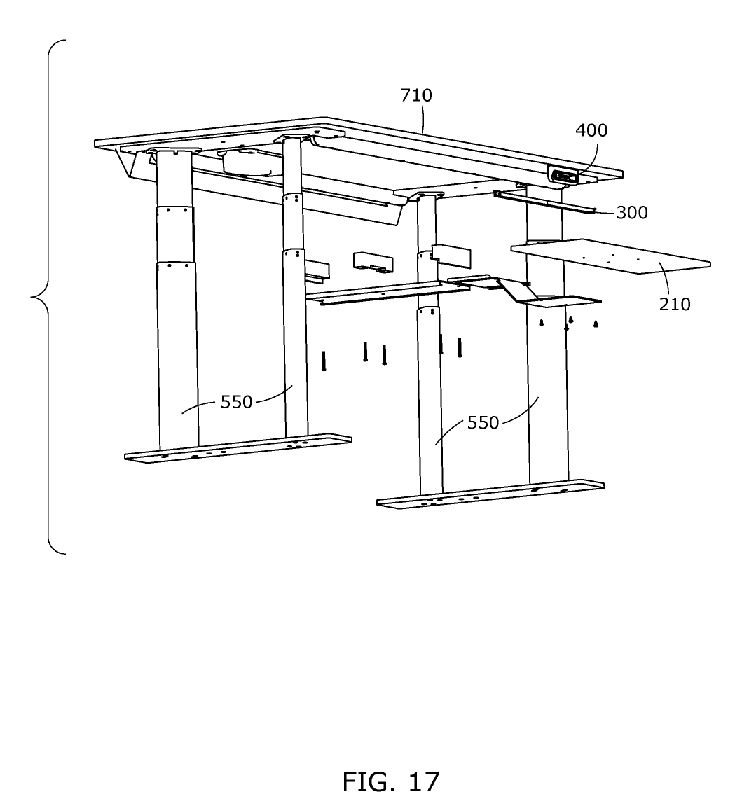

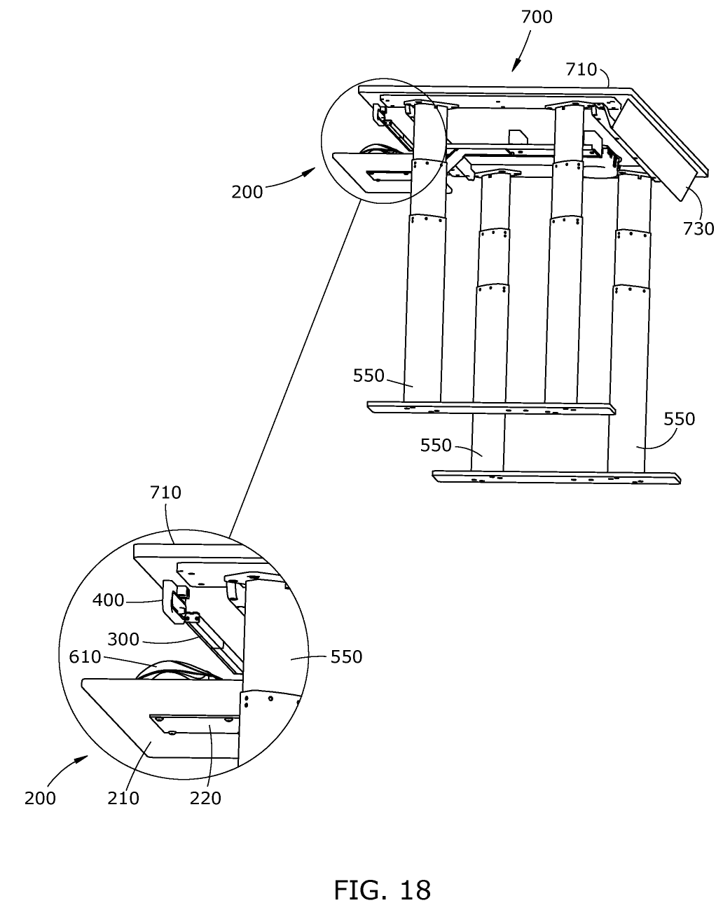

FIGS. 1-18 illustrate two exemplary embodiments of the present disclosure: a two legged height adjustable desk 100 (FIGS. 1-9); and a four legged height adjustable desk 700 (FIGS. 10-18). The desk 100 further comprises two telescopic desk legs 500 that expand to raise the desktop 110 to a standing position, and retract to lower it to a sitting position. The desktop 110 is a flat rectangular member that is oriented parallel to the ground in all positions for sitting and standing, and any height in between the two.

The desktop 110 may further comprise a cable cover 130 attached to and extending vertically downward from the front side of the desktop 110. The purpose of the cable cover is to house electric cables for office equipment residing on top of the desktop 110, such as computer cables and light fixture cables. In the exemplary embodiment of FIGS. 1-9, the cable cover 130 is an open-ended or close-ended rectangular housing extending the length of the desktop front side.

Similarly, desk 700 further comprises four telescopic desk legs 550 that expand to raise the desktop 710 to a standing position, and retract to lower it to a sitting position. The desktop 710 may have a similar shape, size and orientation as the desktop 110. Desk 700 may further comprise a cable cover 730 extending vertically downward from the desktop underside. In the exemplary embodiments of FIGS. 10-18, the cable cover 730 is an open-ended or a close-ended triangular housing extending the length of the desktop front side.

In all embodiments of the present disclosure, the height adjustable desk 100 and 700 further comprises: an extendable-retractable keyboard tray system 200; an ultraviolet (UV) light disinfectant unit 300; and a user control panel 400. Desk 100, 700 may further comprise a motion sensor device (not shown) that is co-located with the UV light unit 300, or located elsewhere on the desk, e.g. the top of the desktop 110.

### Keyboard Tray System

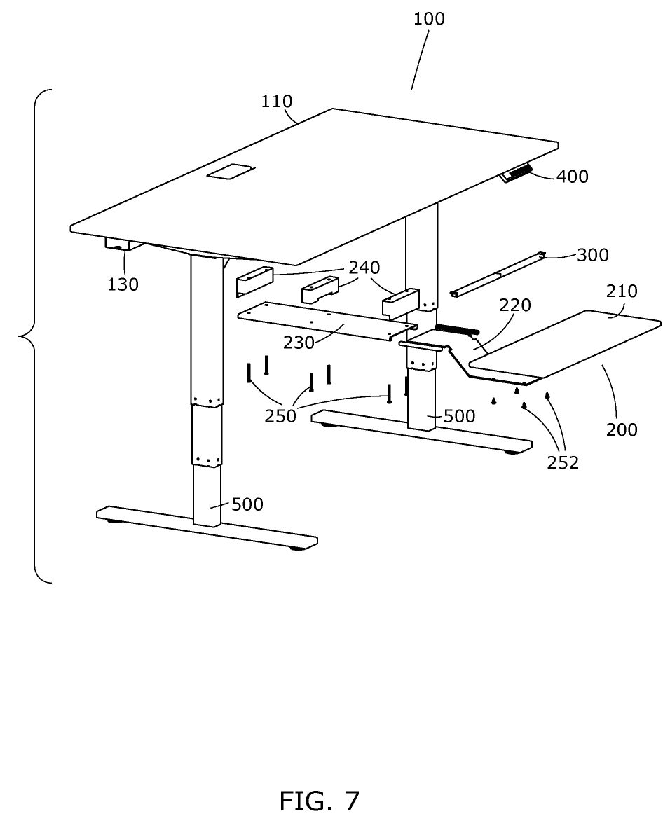

As best illustrated in the exploded FIGS. 7, 8, 16 and 17, the keyboard tray system 200 comprises: a keyboard tray 210 upon which the keyboard 600, mouse 610 and mouse pad rest (see FIG. 1); a bent arm support member 220 rigidly affixed to the underside of the keyboard tray 210 on a backend; a keyboard tray guide rail 230 that the member 220's front end slides within; and a plurality of fixation mechanism(s) 240 that affix the guide rail 230 to the underside of the desktop 110. In the exemplary embodiments of, for example FIGS. 7, 8, 16 and 17, the plurality of fixation mechanism comprises: 1) three blocks 240 that are a few inches in width and are the length of the guide rail 230, and 2) with one each of mechanisms 240 positioned perpendicular to the front, middle, and back end of the rail 230, and 3) affixed between the rail 230 and the desktop 110 underside.

As illustrated in the exemplary embodiments of the figures, the bent arm support member 220 comprises two diagonally positioned, parallel flat plates spaced approximately four to six inches apart, one diagonally above and towards the desktop front side, and one below and towards the desktop backside. The two parallel flat plates are also attached to each other via a third flat plate positioned between and connecting the two flat plates, and oriented at about a forty-five degree angle to them. The three plates may be manufactured as one plate with a bent mid-section (i.e. the third flat plate).

Other embodiments of the member 220 are envisioned as long as the member 220 is able to maintain the keyboard tray 210 in a stable position parallel to the ground, and is able to slide the tray 210 from an extended position suitable for typing to a retracted position fully stored beneath the desktop 110. For example, the bent arm support member 220 may instead comprise a three plate member with the top and bottom plate parallel relative to each other, and connected via a middle third plate perpendicular to both. The top end of the middle plate is rigidly attached to the backend of the top plate and the bottom end of the middle plate is rigidly attached to the frontend of the bottom plate.

In all embodiments, the top plate may further comprise mechanisms to assist in sliding the member 220 through the guide rail 230, such as the exemplified right and left side extenders.

### Ultraviolet (UV) Light Disinfectant Unit

The UV light unit 300 of the present disclosure comprises any UV light source that is mounted on the desk 100, 700 and able to effectively eradicate microbial agents growing on the top side of the keyboard tray 210. Therefore, the unit 300 may be any shape and size and located anywhere on the desk 100, 700 so that its UV rays are directed onto the topside of the keyboard tray 210, but not onto areas surrounding tray 210.

In one embodiment, this directed UV light is only activated when the tray 210 is fully retracted beneath the desktop 110, 710. In other embodiments, the directed UV light is activated when the tray 210 is in any position as long as the user is not near the backside of the desk 100, 700 (i.e. a motion sensor detects when a user is near).

One of skill in the art would know how to adjust the magnitude and duration per the type of UV light unit 300 to effectively eradicate microbial agents on the keyboard tray. In one embodiment, the ultraviolet light unit 300 irradiates UV-C rays that are less than or equal to 280 nanometers. UV-C light emitted may also be set for about 265 nanometers and/or for about 185 nanometers, both of which have been shown to be the most effective at eradicating microbial agents.

The UV light unit 300 may be on for a continuous period or repeatedly pulsed. Durations of exposure are dependent upon the type of UV light unit 300 device (e.g. manufacturer, wavelength, continuous, pulsed, etc.) and should be operated in accordance with manufacturer's instruction. Durations of exposure may range from seconds to minutes, for example about 5 seconds (Umezawa et al) to about 10 minutes (Nerandzic et al).

In the exemplary embodiments of FIGS. 1-18, the UV light unit 300 is a thin rectangular UV light emitting unit that is rigidly mounted onto the underside of the desktop 110, 710 at the edge of the desktop's backside. Unit 300 is centered over the keyboard tray 210, and with a length of between about as long as, or about two-thirds the length of, the tray 210.

Means of activating the UV light unit 300 may comprise one or more of the following. The user may turn on/off the unit 300 with a manual activation switch, such as one on the user control panel 400 or on the side of the UV light unit 300. The user would stand away from the keyboard tray 210 to prevent exposure, such as standing to the right of the desk 100, 700 to easily reach the right side user control panel 400 while the keyboard tray 210 is fully retracted underneath of the desktop 110, 710.

Additionally, or alternatively, means for activating the UV light unit 300 may comprise automated mechanisms attached to the unit 300. For example, unit 300 may be connected to and under the operational control of a motion sensor that detects when a user is not near the keyboard tray 210. The UV light unit 300 could also be programmed to activate on pre-set conditions, such as whenever the keyboard tray 210 is returned to the retracted position.

And, additionally or alternatively, activation mechanisms may also comprise a timer that is set by the user for a one time use, or pre-set for regular scheduled irradiation (e.g. every morning at 3:00 am for 10 minutes).

### User Control Panel

In the various embodiments, the user control panel 400 comprises one activation/deactivation power switch for a user to turn on/off the motors that extend and retract the telescopic desk legs 500, 550.

In another embodiment, the user control panel 400 comprises at least two activation/deactivation power switches: one for raising/lowering the desk legs 500, 550; and one for turning the UV light unit 300 on/off.

The user control panel 400 may further comprise a third power switch to turn a motion sensor on/off. Again, the motion sensor may be located anywhere on or near the backside of the desktop as long as it is able to detect the user behind the desk and near the keyboard tray 210. For example, the panel 400 may be have an activation switch for a motion sensor device that is located on the top surface of the desktop 110, 710 that can communicate wirelessly with, or is wired to, the UV light unit 300 and with the panel 400.

The user control panel 400 is also easily accessible to the user, such as illustrated in FIGS. 1-18, by being located on the right backside of the desktop 110, 710. It is understood that the user control panel 400 may be located elsewhere, such as on the left backside of the desktop for left handed users, or on top of the desktop 110, 710 while within easy arms reach when sitting behind the desk.

Although the present disclosure has been fully described by way of example with reference to the accompanied figures-drawings, it is to be understood that various changes and modifications will be apparent to those of ordinary skill in the art. Therefore, unless such changes and modifications depart from the scope of the present disclosure, then they should be construed as being included within the various embodiments.

### REFERENCES

Nerandzic et al, “Evaluation of a pulsed xenon ultraviolet disinfection system for reduction of healthcare-associated pathogens in hospital rooms”, Infect Control Hosp Epidemiol, 2015 February; 36(2): 192-7.
Nwankiti et al., “Computer Keyboard and Mouse: Etiological Agents for Microbial Infection”, Nature and Science, 2012, 10(10): 162-166.
Pope et al, “Spine Ergonomics”, Annual Review of Biomedical Engineering, Vol. 4: 49-68.
Pronk et al., “Reducing Occupational Sitting Time and Improving Worker Health: The Take-a-Stand Project, 2011”, Centers for Disease Control and Prevention, 2012, Oct. 11; Vol. 9.
Umezawa et al, “A comparative study of the bactericidal activity and daily disinfection housekeeping surfaces by a new portable pulsed UV radiation device”, Curr Microbiol, 2012 June; 64(6): 581-7.

What is claimed is:

1-20) (canceled)

21) A height adjustable desk with a keyboard tray disinfectant unit, the desk comprising:
a) a desktop;
b) a plurality of telescopic height adjustable desk legs;
c) a keyboard tray; and
d) an ultraviolet light unit configured to irradiate at least a portion of the keyboard tray.

22) The height adjustable desk of claim 21 further comprising:
a) a bent keyboard support member affixed to said keyboard tray;
b) a keyboard guide rail affixed to an underside of said desktop;
c) wherein said bent keyboard support member is disposed within said keyboard guide rail;
d) wherein said bent keyboard support member is configured to slide within said keyboard guide rail between a first position wherein said keyboard tray is disposed beneath said desktop and a second position wherein said keyboard tray is extended from said desktop.

23) The height adjustable desk of claim 22, wherein said ultraviolet light unit is connected to an underside of said desktop.

24) The height adjustable desk of claim 23, wherein said ultraviolet light unit is disposed above said keyboard tray.

25) The height adjustable desk of claim 24 wherein said ultraviolet light unit is configured to activate only when said keyboard support member is disposed in said first position.

26) The height adjustable desk of claim 21 further comprising a telescopic leg switch configured to extend said height adjustable desk legs and retract said height adjustable desk legs.

27) The height adjustable desk of claim 21, further comprising an ultraviolet light unit power control switch.

28) The height adjustable desk of claim 21, further comprising a timer connected to said ultraviolet light unit.

29) The height adjustable desk of claim 21 further comprising a motion sensor configured to deactivate said ultraviolet light unit when motion is detected.

30) The height adjustable claim as in claim 29 further comprising a motion sensor power control switch.

31) The height adjustable desk of claim 30 further comprising:
a) a bent keyboard support member affixed to said keyboard tray;
b) a keyboard guide rail affixed to an underside of said desktop;
c) wherein said bent keyboard support member is disposed within said keyboard guide rail;
d) wherein said bent keyboard support member is configured to slide within said keyboard guide rail between a first position wherein said keyboard tray is disposed beneath said desktop and a second position wherein said keyboard tray is extended from said desktop.

32) The height adjustable desk of claim 31, wherein said ultraviolet light unit is connected to an underside of said desktop.

33) The height adjustable desk of claim 32, wherein said ultraviolet light unit is disposed above said keyboard tray.

34) The height adjustable desk of claim 33 wherein said ultraviolet light unit is configured to activate only when said keyboard support member is disposed in said first position.

35) The height adjustable desk of claim 34, further comprising an ultraviolet light unit power control switch.

36) The height adjustable desk of claim 35, further comprising a timer connected to said ultraviolet light unit.

37) The height adjustable desk of claim 36 further comprising a telescopic leg switch configured to extend said height adjustable desk legs and retract said height adjustable desk legs.

38) A method for disinfecting a keyboard tray on a desk comprising
a) providing a desk comprising
i) a desktop;
ii) a plurality of telescopic height adjustable desk legs;
iii) a keyboard tray; and
iv) an ultraviolet light unit configured to irradiate at least a portion of the keyboard tray
b) activating said ultraviolet light unit;
c) deactivating said ultraviolet light unit.

39) The method as in claim 38 wherein said desk further comprises a motion detector, said method further comprising detecting motion by said motion detector.

40) The method as in claim 38 wherein said desk further comprises a timer, wherein activating said ultraviolet light unit is performed at a predetermined time established by said timer.
